(19) United States
(12) Patent Application Publication
(10) Pub. No.: US 2026/0069307 A1
(43) Pub. Date: Mar. 12, 2026

(54) ULTRASONIC TREATMENT DEVICES

(71) Applicant: OLYMPUS MEDICAL SYSTEMS CORP., Tokyo (JP)

(72) Inventors: Tsubasa NIIYAMA, Tokyo (JP); Koichi TSURUTA, Yokohama-shi (JP); Kazuhiro MORISAKI, Yokohama-shi (JP); Fumiya ISHIKAWA, Hachioji-shi (JP); Marina ASAHINA, Hachioji-shi (JP); Hidenosuke HASE, Hachioji-shi (JP); Tsunetaka AKAGANE, Hachioji-shi (JP); Ojiro KITAMURA, Hachioji-shi (JP); Kazue TANAKA, Sagamihara-shi (JP)

(73) Assignee: OLYMPUS MEDICAL SYSTEMS CORP., Tokyo (JP)

(21) Appl. No.: 19/393,889

(22) Filed: Nov. 19, 2025

Related U.S. Application Data
(62) Division of application No. 17/591,079, filed on Feb. 2, 2022.
(60) Provisional application No. 63/152,380, filed on Feb. 23, 2021.

(51) Int. Cl. A61B 17/32; A61B 17/28

(52) U.S. Cl. CPC A61B 17/320092; A61B 17/282; A61B 2017/2825; A61B 2017/320069; A61B 2017/320094

(57) ABSTRACT

Ultrasonic treatment device includes a blade configured to apply ultrasonic vibrations to target tissue and a jaw movable relative to the blade between open and closed configurations. In some examples, the jaw includes a base having an inner channel and a plurality of slots in communication with the inner channel, and a pad having a plurality of protrusions, each protrusion configured to be received within a different slot of the plurality of slots. In further examples, the jaw includes a base having a pair of side walls defining an inner channel, one of the side walls including a rail member extending into the channel, and a pad having a single groove for receiving the rail member when the pad is positioned within the channel. A surface of the groove is configured to contact a surface of the rail when a force is applied against a grasping surface of the pad.

### RELATED APPLICATION DATA

This application is a divisional application of U.S. application Ser. No. 17/591,079, filed Feb. 2, 2022, which is based on and claims priority under 35 U.S.C. § 119 to U.S. Provisional Application No. 63/152,380, filed on Feb. 23, 2021, the entire contents of each of these applications is incorporated herein by reference.

### FIELD

The present disclosure, according to some embodiments, relates to ultrasonic treatment devices for use in surgically treating a tissue. More particularly, in some embodiments the present disclosure relates to ultrasonic surgical devices having a grasping instrument for clamping onto a tissue to be treated. In further embodiments, the present disclosure provides a grasping instrument for an ultrasonic treatment device with improved thermal management.

### BACKGROUND

Ultrasonic treatment devices are configured to utilize ultrasonic mechanical vibrations to surgically treat various medical conditions. Ultrasonic vibrations can be used, for example, to cut, dissect, and/or cauterize soft tissue of a patient. Such ultrasonic treatment devices may generally include a tissue-contacting member for applying ultrasonic vibrations to the tissue to be treated, an ultrasonic transducer for converting electric energy into ultrasonic vibrations, and a transmission element for transmitting the ultrasonic vibrations from the ultrasonic transducer to the tissue-contacting member.

In some ultrasonic treatment devices, the tissue-contacting member may be a single-component instrument, for example, a blade, ball coagulator, or hook for applying the ultrasonic vibrations to the tissue. In other ultrasonic treatment devices, the tissue-contacting member includes a multi-component instrument, for example, a grasping instrument having a blade for applying the ultrasonic vibrations to the tissue, and a jaw capable of pivoting relative to the blade such that the tissue can be clamped between the blade and the jaw. The jaw may be configured to apply a compressive force against the tissue while the ultrasonic vibrations are applied by the blade, allowing for faster cutting and/or coagulation in some instances.

The jaw of the grasping instrument may include a pad for pressing against the tissue. The pad may be composed of a polymer material, for example, polytetrafluoroethylene (PTFE) or other polymer resin, and may have some flexibility. The pad includes a surface which is intended to contact the tissue when the jaw clamps against the tissue. This pad surface may also come into contact with the vibrating blade during use, for example, once the blade has cut through the tissue. However, this contact between the pad and the vibrating blade can result in excessive heat due to friction, potentially causing damage to the pad.

### SUMMARY OF THE INVENTION

The present disclosure, according to some embodiments, provides an ultrasonic treatment device having improved heat dissipation between a pad and a base. In some embodiments, an ultrasonic treatment device according to the present disclosure includes a blade configured to apply ultrasonic vibrations to a target tissue, and a jaw that is movable with respect to the blade between an open configuration and a closed configuration. In some embodiments, the jaw includes a base having an inner channel, and a plurality of slots in communication with the inner channel. In some embodiments, the jaw further includes a pad positionable within the channel of the base, the pad having a grasping surface configured to contact the target tissues, and plurality of protrusions. The protrusions may extend from a portion of the pad that is opposite of the grasping surface.

In some embodiments, each protrusion is configured to be received within a different slot of the plurality of slots when the pad is positioned within the inner channel. In some embodiments, the pad includes at least one groove, and the base includes at least one rail member configured to be received in the at least one groove. In some embodiments, each protrusion of the pad includes a groove, and each slot includes a rail member configured to be received in the groove of one of the protrusions. In some embodiments, the at least one groove is defined in part by a bearing surface configured to contact the at least one rail member when a force is applied against the grasping surface of the pad. In some embodiments, the pad is moveable from a first position, in which the bearing surface is not in contact with the rail member, to a second position, in which the bearing surface is in contact with the rail member. In some embodiments, the pad is moveable from the first position to the second position in response to the force being applied against the grasping surface of the pad. The force may be applied, for example, by the blade when the jaw is in the closed configuration.

In some embodiments, the inner channel is defined in part by a wall of the base, for example, a side wall or a top wall. In some embodiments, the wall includes at least one heat sink configured to contact the pad. In some embodiments, the at least one heat sink extends into the inner channel and is configured to contact a portion of the pad located between two protrusions of the plurality of protrusions. In some embodiments, the at least one heat sink is configured to contact a surface of the pad that is opposite of the grasping surface. In some embodiments, the wall includes a plurality of heat sinks, each heat sink being located between different pairs of slots of the plurality of slots. In some embodiments, the at least one heat sink includes a portion of the wall having a thickness greater than a thickness of an adjacent portion of the wall.

In some embodiments, the pad does not have a constant cross-sectional shape along a majority of a length of the pad. For example, in some embodiments, the pad may have different heights along the length of the pad, e.g., a first height at the protrusions, and a second height between the protrusions. In some embodiments, the cross-sectional shape of the pad may be symmetric along a majority of a length of the pad. In further embodiments, the base includes a plurality of openings, each opening communicating with one of the plurality of slots. The plurality of openings may be located on one or more side walls of the base.

In further embodiments, the present disclosure provides methods for assembling or manufacturing ultrasonic treatment devices, or components thereof. In some such embodiments, a method for manufacturing an ultrasonic treatment device, or a component thereof, includes providing a base having an inner channel, and at least one slot in communication with the inner channel, and positioning a pad at least partially into the inner channel, the pad having a grasping surface for contacting a target tissue, and at least one protrusion opposite of the grasping surface. In some embodiments, the method further includes inserting the at least one protrusion into the at least one slot by moving the pad relative to the base in a first direction, and moving the pad relative to the base in a second direction that is different from the first direction while the at least one protrusion is inserted into the slot. In some embodiments, the pad includes at least one groove, and the base includes at least one rail member. In some embodiments, moving the pad relative to the base in the second direction causes the at least one rail member to be received within the at least one groove. In some embodiments, the first direction is oblique or perpendicular to a longitudinal direction of the base. In some embodiments, the second direction is parallel to the longitudinal direction of the base. In some embodiments, moving the pad relative to the base in the first direction inserts the at least one protrusion into a proximal portion of the at least one slot, and moving the pad relative to the base in the second direction causes the at least one protrusion to move to a distal portion of the at least one slot. In some embodiments, the pad includes a plurality of protrusions, and the base includes a plurality of slots, each protrusion being configured to be inserted into a different slot of the plurality of slots. In some embodiments, the base and pad may be pivotably coupled to a blade configured apply ultrasonic vibrations to a target tissue.

In some further embodiments, an ultrasonic treatment device includes a blade configured to apply ultrasonic vibrations to a target tissue, and a jaw that is movable with respect to the blade between an open configuration and a closed configuration, the jaw including a base having an inner channel defined at least partially by a first side wall and a second side wall, the first side wall including at least one rail member extending into the inner channel. In some embodiments, only the first side wall includes the at least one rail member. In some embodiments, the jaw further includes a pad having a grasping surface for contacting the target tissue, and a single groove for receiving the at least one rail member when the pad is positioned within the channel. In some embodiments, the groove is defined in part by a bearing surface configured to contact the at least one rail member when a force is applied against the grasping surface of the pad. The force may be applied, for example, by the blade when the jaw is in the closed configuration. In some embodiments, the pad may have a cross-sectional shape that is asymmetrical along a majority of a length of the pad.

In some embodiments, the base further includes one or more side openings that communicate with the inner channel, the one or more side openings being located on only one of the first and second side walls. In some embodiments, the one or more side openings are located only on the first side wall that includes the at least one rail member. In some embodiments, the at least one rail member extends from the first side wall below the one or more side openings. In some embodiments, the first side wall includes a separate rail member below each of the one or more side openings. In some embodiments, the first side wall further includes at least one heat sink extending into the inner channel. The at least one heat sink may be positioned and configured to contact the pad. In some embodiments, the first side wall includes a plurality of rail members, and the at least one heat sink is located between two rail members of the plurality of rail members. In some embodiments, the at least one rail member extends from the at least one heat sink. In some embodiments, the at least one heat sink is formed from a portion of the first side wall having a thickness greater than a thickness of the second side wall. In some embodiments, the base further includes a top wall having one or more top openings that communicate with the inner channel. In some embodiments, the at least one heat sink borders the one or more top openings. The at least one heat sink, in some embodiments, serves to dissipate heat away from the pad.

### BRIEF DESCRIPTION OF THE DRAWINGS

The foregoing summary, as well as the following detailed description, will be better understood when read in conjunction with the appended drawings. For the purpose of illustrating the present disclosure, there are shown in the drawings embodiments which are presently preferred, wherein like reference numerals indicate like elements throughout. It should be noted, however, that aspects of the present disclosure can be embodied in different forms and thus should not be construed as being limited to the illustrated embodiments set forth herein. The elements illustrated in the accompanying drawings are not necessarily drawn to scale, but rather, may have been exaggerated to highlight the important features of the subject matter therein. Furthermore, the drawings may have been simplified by omitting elements that are not necessarily needed for the understanding of the disclosed embodiments.

FIG. 1A is a perspective view of an example ultrasonic treatment device having a grasping instrument shown in an open configuration according to an embodiment of the present disclosure.

FIG. 1B is an enlarged view of the grasping instrument of the ultrasonic treatment device of FIG. 1A.

FIG. 2 is a generalized diagram illustrating certain components of the ultrasonic treatment device of FIG. 1A.

FIG. 3 is a perspective view of a grasping instrument of an ultrasonic treatment device having a jaw and a blade in a closed configuration according to an embodiment of the present disclosure;

FIGS. 4A-4G illustrate views of a base and pad of a jaw according to one embodiment. FIG. 4A shows a perspective view of the base and pad received therein. FIG. 4B shows a side view of the base and pad. FIG. 4C shows a top view of the base and pad. FIG. 4D shows a cross-sectional view of the base and pad along the plane designated by line 4D-4D in FIG. 4C. FIG. 4E shows a cross-sectional view of the base and pad along the plane designated by line 4E-4E in FIG. 4C. FIG. 4F shows a cross-sectional view of the base and pad along the plane designated by line 4F-4F in FIG. 4C. FIG. 4G shows a cross-sectional view of the base and pad along the plane designated by line 4G-4G in FIG. 4C.

FIG. 5 is a bottom view of the base shown in FIGS. 4A-4G showing areas that contact the pad.

FIG. 6 is a cross-sectional view of a base and pad according to certain embodiments of the present disclosure illustrating increased contact between the pad and the base.

FIGS. 7A-7F illustrate views of a base and pad of a jaw according to certain embodiments of the present disclosure. FIG. 7A shows a side view of the base and pad received therein. FIG. 7B shows a top view of the base and pad. FIG. 7C shows a cross-sectional view of the base and pad along the plane designated by line 7C-7C in FIG. 7B. FIG. 7D shows a cross-sectional view of the base and pad along the plane designated by line 7D-7D in FIG. 7B. FIG. 7E shows a cross-sectional view of the base and pad along the plane designated by line 7E-7E in FIG. 7B. FIG. 7F shows a cross-sectional view of the base and pad along the plane designated by line 7F-7F in FIG. 7B.

FIG. 8 is a bottom view of the base shown in FIGS. 7A-7F showing areas that contact the pad.

FIG. 9A-9D illustrate steps for assembling the pad and the base shown in FIGS. 7A-7F.

FIGS. 10A-10E illustrate views of a base and pad of a jaw according to further embodiments of the present disclosure. FIG. 10A shows a side view of the base and pad received therein. FIG. 10B shows a top view of the base and pad. FIG. 10C shows a cross-sectional view of the base and pad along the plane designated by line 10C-10C in FIG. 10B. FIG. 10D shows a cross-sectional view of the base and pad along the plane designated by line 10D-10D in FIG. 10B. FIG. 10E shows a cross-sectional view of the base and pad along the plane designated by line 10E-10E in FIG. 10B.

FIG. 11 is a bottom view of the base shown in FIGS. 10A-10E showing areas that contact the pad.

FIGS. 12A-12F illustrate views of a base and pad of a jaw according to certain embodiments of the present disclosure. FIG. 12A shows a side view of the base and pad received therein. FIG. 12B shows a top view of the base and pad. FIG. 12C shows a cross-sectional view of the base and pad along the plane designated by line 12C-12C in FIG. 12B. FIG. 12D shows a cross-sectional view of the base and pad along the plane designated by line 12D-12D in FIG. 12B. FIG. 12E shows a cross-sectional view of the base and pad along the plane designated by line 12E-12E in FIG. 12B. FIG. 12F shows a cross-sectional view of the base and pad along the plane designated by line 12F-12F in FIG. 12B.

FIG. 13 is a bottom view of the base shown in FIGS. 12A-12F showing areas that contact the pad.

### DETAILED DESCRIPTION

The present subject matter will now be described more fully hereinafter with reference to the accompanying Figures, in which representative embodiments are shown. The present subject matter can, however, be embodied in different forms and should not be construed as limited to the embodiments set forth herein. Rather, these embodiments are provided to describe and enable one of skill in the art. All publications, patent applications, patents, and other references mentioned herein are incorporated by reference in their entirety.

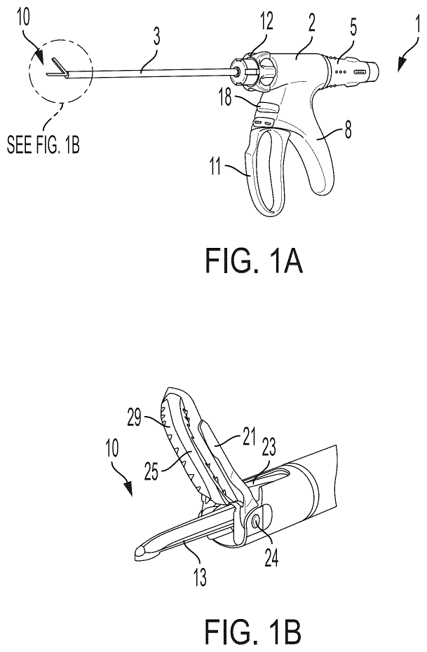

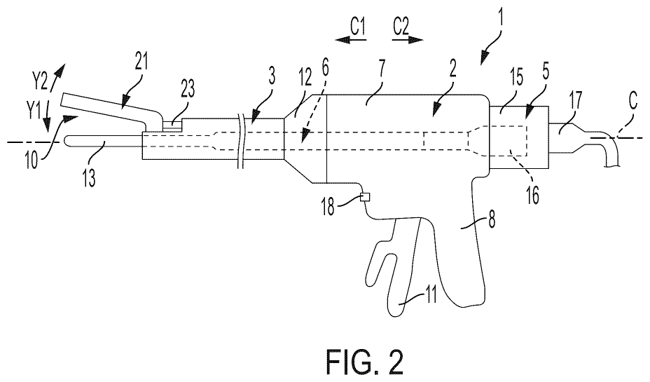

FIGS. 1A-2 illustrate an ultrasonic treatment device 1, according to some embodiments of the present disclosure, which may be used to treat certain medical conditions in a patient. More particularly, FIG. 1A provides a perspective view of ultrasonic treatment device 1, which in some embodiments includes a housing 2 and a grasping instrument 10 located at a distal end of ultrasonic treatment device 1. In some embodiments, grasping instrument 10 is connected to housing 2 by an elongate shaft 3. Housing 2, in some embodiments, includes a grip 8 configured to be held in the hand of a user (e.g., surgeon or other operator), and a handle 11 that is movable relative to grip 8. In some embodiments, grasping instrument 10 is configured to open and close by movement of handle 11 relative to grip 8. For example, in some embodiments, moving handle 11 away from grip 8 causes grasping instrument 10 to open, while moving handle 11 toward grip 8 causes grasping instrument 10 to close. In other embodiments, moving handle 11 away from grip 8 causes grasping instrument 10 to close, while moving handle 11 toward grip 8 causes grasping instrument 10 to open. As further shown in FIG. 1A, ultrasonic treatment device 1 may also include a rotary member 12 for rotating shaft 3 and grasping instrument 10, a transducer unit 5 including an ultrasonic transducer, and one or more operation buttons 18 for operating the ultrasonic transducer, details of which will be further described herein.

FIG. 1B provides an enlarged view of grasping instrument 10, according to certain embodiments. In some embodiments, grasping instrument 10 generally includes a blade 13 that is configured to apply ultrasonic vibrations to a target tissue, and a jaw 21 that is movable relative to blade 13 between an open configuration (an example of which is shown) and a closed configuration. In the open configuration, jaw 21 is rotated or pivoted away from blade 13. In the closed configuration, jaw 21 is rotated or pivoted toward blade 13. In some embodiments, transducer unit 5 is configured to cause blade 13 to mechanically vibrate during use at a frequency that is sufficient for treating the target tissue (e.g., at an ultrasonic frequency). Blade 13 may be configured to cut or dissect the target tissue, though blade 13 is not necessarily limited to this use. For example, in some embodiments, blade 13 may be a tool configured for ultrasonic coagulation.

In some embodiments jaw 21 is configured to press the target tissue against blade 13 during operation of ultrasonic treatment device 1. In some embodiments, jaw 21 includes a pad 25 that is configured to contact the target tissue, and a base 29 for holding pad 25. Pad 25 may be composed of a polymer material, for example, polytetrafluoroethylene (PTFE) or other polymer resin. Base 29, in some embodiments, may be made from metal or metal alloy. In some embodiments, jaw 21 is rotatably attached relative to blade 13, for example, via a hinge or pivot 24. In further embodiments, jaw 21 is connected to a movable member 23 (e.g., a push rod) that, in turn, is coupled to handle 11. As such, movable member 23 may serve as a mechanical linkage between jaw 21 and handle 11. In some such embodiments, movement of handle 11 (e.g., relative to grip 8 or housing 2) causes movable member 23 to rotate jaw 21 about hinge or pivot 24 in order to open or close grasping instrument 10.

FIG. 2 is a diagram showing additional components of ultrasonic treatment device 1, according to some embodiments. As shown in the illustrated embodiment of FIG. 2, ultrasonic treatment device 1 includes housing 2, shaft 3, transducer unit 5, as well as a rod member 6 extending through shaft 3 and connected to blade 13. In some embodiments, shaft 3 has a longitudinal axis C as a center axis. One side in a direction along the longitudinal axis Cis a distal side (arrow C1 side), and a side opposite to the distal side is a proximal side (arrow C2 side). Shaft 3 extends along the longitudinal axis C.

Housing 2 is configured to be hand-held (e.g., by a surgeon or other medical practitioner), and is coupled to the proximal side of the shaft 3. In some embodiments, housing 2 includes a housing body 7 which extends along the longitudinal axis C, and grip 8 extends from the housing body 7 in a direction crossing the longitudinal axis C. In addition, in some embodiments, handle 11 is movably attached to the housing 2. In some embodiments, handle 11 is configured to rotate relative to housing 2 around an attachment position. The attachment position may be located on or within housing 2. In some embodiments, handle 11 is able to move or rotate toward or away from grip 8. In the illustrated embodiment, handle 11 is located on the distal side with respect to the grip 8, and the direction of movement of the handle 11 relative to the grip 8 may be substantially parallel to the longitudinal axis C, but this is not restrictive. In another embodiment, the handle 11 may be provided on the proximal side with respect to the grip 8. In a further embodiment, the handle 11 and grip 8 may be provided on mutually opposite sides with respect to the longitudinal axis C as the center, and the direction of movement of the handle 11 relative to the grip 8 may be substantially perpendicular to the longitudinal axis C.

In some embodiments, a rotary member (e.g., rotary knob) 12 is attached to the housing body 7 from the distal side. Shaft 3 may be inserted into the inside of the rotary member 12 from the distal side. In some embodiments, shaft 3 is fixed to the rotary member 12, and is rotatable together with the rotary member 12 around the longitudinal axis C relative to the housing 2. In some such embodiments, this allows grasping instrument 10 to rotate with respect to housing 2 about longitudinal axis C.

In some embodiments, rod member 6 is configured to transmit ultrasonic vibration from transducer unit 5 to blade 13, and extends from the inside of the housing 2 toward the distal side along the longitudinal axis C through the inside of shaft 3. A distal portion of the rod member 6 is provided with blade 13. Blade 13 and rod member 6 may be of unitary construction according to some embodiments. In other embodiments, blade 13 is a separate component from rod member 6 that is attached to the distal portion of rod member 6. Rod member 6 is inserted through the shaft 3 in such a state that the blade 13 projects from the distal end of the shaft 3 toward the distal side.

In some embodiments, transducer unit 5 includes a transducer case 15 and an ultrasonic transducer 16. Transducer case 15 is attached to the housing body 7 from the proximal side. In addition, one end of a cable 17 is connected to the transducer case 15. Cable 17, in some embodiments, is a power cable configured to transmit electrical energy to ultrasonic treatment device 1, e.g., to power ultrasonic transducer 16. In some embodiments, another end of the cable 17 is detachably connected to an energy control device (not shown), for example, a power supply. The ultrasonic transducer 16 is disposed in the inside of the transducer case 15. In some embodiments, the ultrasonic transducer 16 extends along the longitudinal axis C. In some embodiments, ultrasonic transducer 16 is connected to the rod member 6 at a proximal portion of rod member 6.

In some embodiments, one or more operation buttons 18 is attached to the housing 2. An operation for outputting electric energy from the energy control device is input by the operation button 18. If the operation is input by the operation button 18, AC electric power of a predetermined frequency, for instance, is supplied as electric energy from the energy control device to the ultrasonic transducer 16 via electric wiring (not shown) which extends in the inside of the cable 17. In some embodiments, the ultrasonic transducer 16 includes a piezoelectric element (not shown) which is configured to convert the electric energy to ultrasonic vibration. The ultrasonic vibration generated by the ultrasonic transducer 16 is transmitted in rod member 6 from the proximal side to the distal side. Further, the ultrasonic vibration is transmitted to blade 13 by rod member 6. In some embodiments, ultrasonic transducer 16 and rod member 6 vibrate at a certain frequency in a predetermined frequency range by transmitting the ultrasonic vibration. In some embodiments, the direction of vibration of the rod member 6 and ultrasonic transducer 16 may be substantially parallel to the longitudinal axis C. In other embodiments, a footswitch or the like, which is separate from ultrasonic treatment device 1, may be provided in place of the operation button 18, or in addition to the operation button 18.

In some embodiments, jaw 21 is rotatably attached to a distal portion of shaft 3. In some embodiments, movable member 23 (e.g., a push rod) extends along or parallel to longitudinal axis C in the inside of shaft 3, and a distal portion of the movable member 23 is connected to jaw 21. In other embodiments, the movable member 23 may extend on the outside of shaft 3, and shaft 3 may extend in the inside of movable member 23. In some embodiments, movable member 23 extends toward the proximal side to the inside of housing 2. In some embodiments, handle 11 is coupled to movable member 23 at a location inside of housing body 7. In some embodiments, moving handle 11 away from or toward grip 8 causes movable member 23 to move along or parallel to longitudinal axis C. This movement of movable member 23 in turn applies a driving force from the movable member 23 to jaw 21, according to some embodiments, causing jaw 21 to rotate about an attachment position to the shaft 3 (e.g., hinge or pivot 24 of FIG. 1B). This allows jaw 21 to open or close relative to blade 13. In some embodiments, by jaw 21 and blade 13 closing relative to each other, a treatment target, such as a biological tissue, can be grasped between the jaw 21 and blade 13. The closing direction (the direction of arrow Y1) and the opening direction (the direction of arrow Y2) of jaw 21 cross the longitudinal axis C. In some embodiments, when jaw 21 and blade 13 are closed relative to each other, the longitudinal direction of jaw 21 becomes substantially parallel to the longitudinal axis C of the shaft 3.

As discussed, grasping instrument 10 includes blade 13 and jaw 21. In some embodiments, grasping instrument 10 and rod member 6 can rotate together with shaft 3 and rotary member 12 relative to housing 2 around the longitudinal axis C. In other embodiments, the rotary member 12 may not be provided, and shaft 3, grasping instrument 10, and rod member 6 may be configured to be unrotatable around the longitudinal axis C, relative to the housing 2.

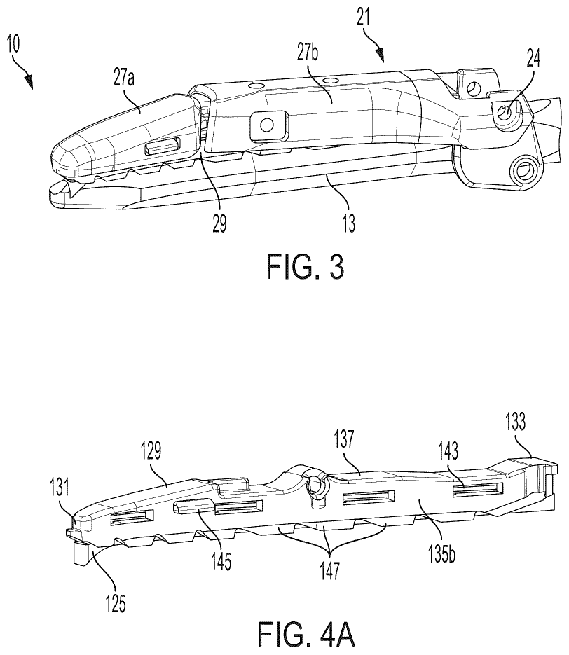

Referring now to FIG. 3, there is shown an isolated perspective view of a grasping instrument 10 according to some embodiments, which may be used with ultrasonic treatment device 1. Grasping instrument 10 of FIG. 3 is shown in a closed configuration, wherein jaw 21 is positioned against blade 13. In some embodiments, jaw 21 includes a cover member that is rotatably coupled relative to blade 13 by a hinge or pivot 24. In some embodiments, cover member includes a distal cover member 27a that is secured to and partially surrounds a distal portion of base 29, and a proximal cover member 27b that is secured to and partially surrounds jaw 21. Base 29, in turn, is configured to hold pad 25 (shown in FIG. 1B).

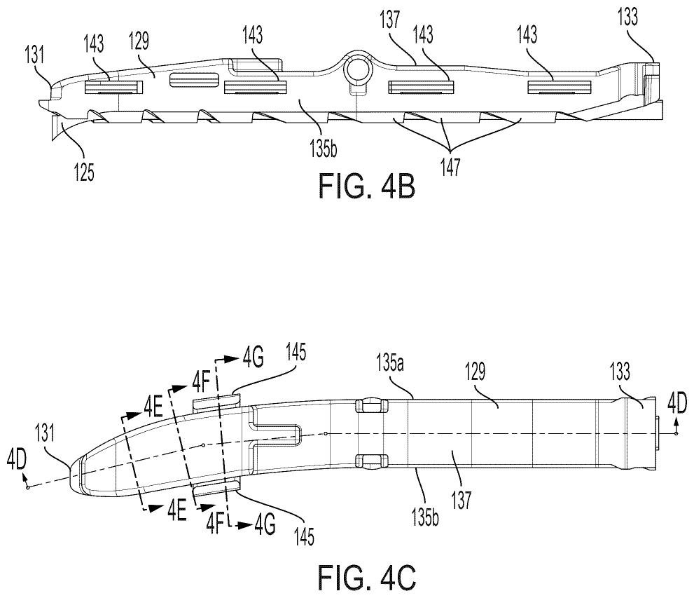

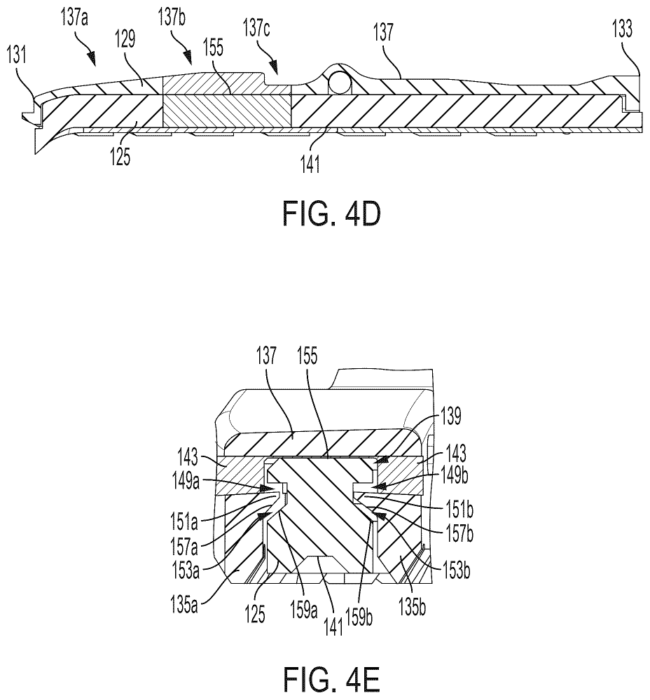

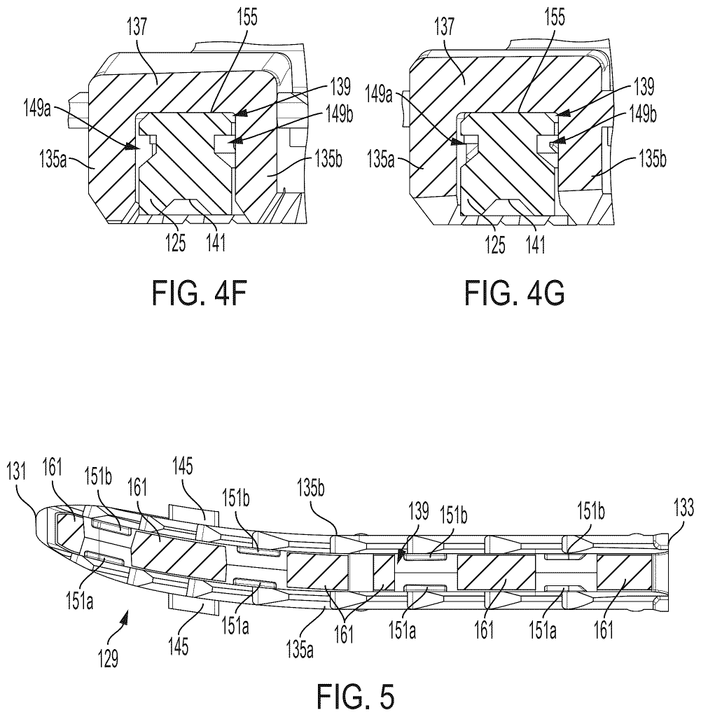

FIGS. 4A-4G illustrate views of a base 129 and pad 125, which may be used with jaw 21 according to one embodiment. In some embodiments, base 129 and pad 125 may be used for base 29 and pad 25 in grasping instrument 10 of ultrasonic treatment device 1. In particular, FIG. 4A shows an isolated perspective view of base 129 and pad 125 received therein. FIG. 4B shows a side view of base 129 and pad 125. FIG. 4C shows a top plan view of base 129 and pad 125. FIG. 4D shows a cross-sectional view of base 129 and pad 125 along the plane designated by line 4D-4D in FIG. 4C. FIG. 4E shows a cross-sectional view of base 129 and pad 125 along the plane designated by line 4E-4E in FIG. 4C. FIG. 4F shows a cross-sectional view of base 129 and pad 125 along the plane designated by line 4F-4F in FIG. 4C. FIG. 4G shows a cross-sectional view of base 129 and pad 125 along the plane designated by line 4G-4G in FIG. 4C.

As shown in the illustrated embodiment, base 129 includes a distal end 131 and a proximal end 133 that is opposite of distal end 131. Base 129, in some embodiments, further includes a pair of opposite side walls 135a, 135b, and a top wall 137 that may extend from distal end 131 to proximal end 133 and includes first portion 137a, second portion 137b and third portion 137c. In some embodiments, side walls 135a, 135b may have substantially the same thicknesses. In some embodiments, base 129 includes a channel 139 (e.g., FIGS. 4E-4G) that is defined by one or more walls such as side walls 135a, 135b, and top wall 137. In some embodiments, channel 139 is sized and configured for receiving pad 125 therein. In some embodiments, channel 139 is open opposite of top wall 137 such that a grasping surface 141 of pad 125 may be exposed while pad 125 is received in channel 139. At least a portion of grasping surface 141, in some embodiments, may be configured to contact the target tissue during use. In some embodiments, grasping surface 141 may also come into contact with blade 13 when grasping instrument 10 is in the closed configuration. Pad 125 further includes a top surface 155 that is opposite grasping surface 141. In some embodiments, top surface 155 may abut against an internal surface of top wall 137. FIG. 5 is a bottom view of base 129 showing portions 161 that may contact the pad according to some embodiments.

Referring again to FIGS. 4A-4G, in some embodiments, base 129 includes one or more openings 143. In some embodiments, each of side walls 135a, 135b include one or more openings 143. As shown in FIGS. 4A and 4B, openings 143 are elongated openings arranged linearly along a longitudinal axis of base 129. In some embodiments, the one or more openings extend through the entire thickness of side wall 135a or 135b and communicate with channel 139, as best shown in FIG. 4E. In some embodiments, base 129 includes one or more external projections 145. In some such embodiments, the one or more external projections 145 are received within openings or recesses on distal cover member 27a of jaw 21 (see FIG. 3) to help secure base 129 to distal cover member 27a. In some embodiments, each of side walls 135a, 135b include at least one external projection 145 that extends from an exterior portion of side wall 135a, 135b. In some embodiments, a bottom edge of each of side walls 135a, 135b may optionally include one or more teeth 147. Teeth 147, in some embodiments, may assist jaw 21 in grasping and holding the target tissue against blade 13 during use.

Pad 125, in some embodiments, is sized to extend from distal end 131 to proximal end 133 of base 129 within channel 139. Pad 125 may be constructed from of a polymer material, for example, polytetrafluoroethylene (PTFE) or other polymer resin, according to some embodiments. In some embodiments, except for certain end portions, pad 125 includes a generally constant cross-sectional shape along the majority of its length. The generally constant cross-sectional shape may further be symmetric (e.g., reflection symmetry) according to some embodiments, as shown in FIGS. 4E-4G.

Pad 125 may form a sliding joint with base 129 according to some embodiments. Pad 125, in some embodiments, includes a pair of grooves 149a, 149b (see, e.g., FIG. 4E) located on opposing lateral sides of pad 125. In some such embodiments, grooves 149a, 149b extend along the entire length or at least a majority of the length of pad 125. In some embodiments, side walls 135a, 135b of base 129 include rail members 151a, 151b that are configured to be received within grooves 149a, 149b of pad 125, respectively. Rail members 151a, 151b, in some embodiments, protrude from internal surfaces of side walls 135a, 135b into channel 139 and are sized to fit within grooves 149a, 149b to form a sliding fit. In some embodiments, rail members 151a, 151b extend substantially along the entire length of side walls 135a, 135b. In other embodiments, side walls 135a, 135b include a plurality of rail members 151a, 151b that are located at different positions along the length of side walls 135a, 135b.

As particularly shown in FIG. 4E, in some embodiments gaps 153a, 153b may exist on the sides of pad 125 between pad 125 and rail members 151a, 151b. More specifically, in some embodiments, gaps 153a, 153b may exist between lower surfaces of rail members 151a, 151b and lower surfaces of grooves 149a, 149b, as illustrated. In some embodiments, gaps 153a, 153b may occur, in some embodiments, because the clearance between top surface 155 of pad 125 and top wall 137 of base 129 is smaller than the distance separating lower surfaces 157a, 157b of rail members 151a, 151b and lower surfaces 159a, 159b of grooves 149a, 149b. In some such embodiments, the lower surfaces 157a, 157b of rail members 151a, 151b are prevented from contacting the lower surfaces 159a, 159b of grooves 149a, 149b because the abutment of top surface 155 of pad 125 against top wall 137 prevents further upward movement of pad 125 relative to base 129.

It has been found that gaps 153a, 153b may decrease the ability for heat to be transferred from pad 125 to base 129 in certain embodiments. As discussed, pad 125 includes a grasping surface 141 which is intended to contact the tissue when jaw 21 clamps against the tissue. This grasping surface 141 may also come into contact with vibrating blade 13 during use, for example, once blade 13 has entirely cut through the tissue. Contact between pad 125 and vibrating blade 13, in some instances, can result in excessive heat accumulating in pad 125 as a result of friction between the two components, particularly at grasping surface 141 of pad 125. This excess heat may, in turn, cause damage to pad 125.

In some embodiments, it may be desirable to reduce the amount of heat that may accumulate in the pad of jaw 21. In some embodiments of the present disclosure, a method for reducing the amount of heat accumulation in the pad includes increasing the contact area between the pad and the base. In some embodiments, increasing the contact area between the pad and the base allows for increased heat transfer from the pad to the base, resulting in greater dissipation of heat from the pad. In some embodiments, a further method for reducing the amount of heat accumulation in the pad includes increasing the heat capacity of the base. In some embodiments, for example, the heat capacity of the base can be increased by increasing the thickness of portions of the base (e.g., portions of the lateral side walls and/or top wall). In some embodiments, portions of the base are increased in thickness without changing the overall dimensions of base. In some embodiments, this can be achieved by modifying the shape of the base as well as the shape of the pad. In some embodiments of the present disclosure, both the contact area between the pad and the base is increased and the heat capacity of the base is increased.

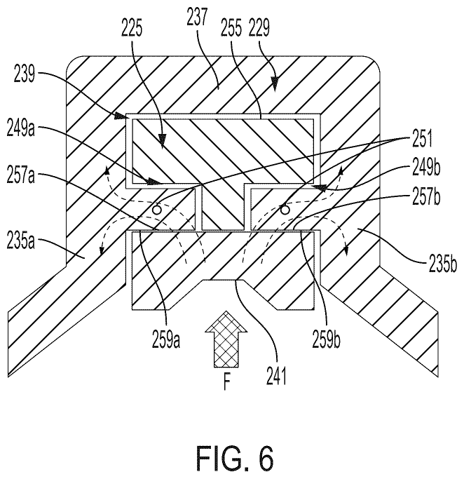

FIG. 6 shows a cross-sectional view of a pad 225 and a base 229 which may be used with the ultrasonic treatment device 1 according to certain embodiments of the present disclosure. In some embodiments, pad 225 and base 229 have increased contact area compared to pad 125 and base 129 of FIGS. 4A-4G. As shown in FIG. 6, base 229 in some embodiments includes a pair of opposite side walls 235a, 235b, and a top wall 237. Base 229 may be constructed from a metal or metal alloy. In some embodiments, side walls 235a, 235b may have substantially the same thicknesses. In some embodiments, base 229 includes an inner channel 239 that is defined by one or more walls such as side walls 235a, 235b, and top wall 237. In some embodiments, channel 239 is sized and configured for receiving pad 225 therein. In some embodiments, channel 239 is open opposite of top wall 237 such that a grasping surface 241 of pad 225 may be exposed while pad 225 is received in channel 239. At least a portion of grasping surface 241, in some embodiments, may be configured to contact the target tissue during use. In some embodiments, grasping surface 241 may also come into contact with blade 13 (not shown). Pad 225 further includes a top surface 255 that is opposite grasping surface 241. In some embodiments, top surface 255 may or may not abut against an internal surface of top wall 237.

Pad 225 may form a sliding joint with base 229 according to some embodiments. In some embodiments, pad 225 and base 229 are arranged in at least one tongue and groove configuration. Pad 225, in some embodiments, includes a pair of grooves 249a, 249b located on opposing lateral sides of pad 225. In some embodiments, side walls 235a, 235b of base 229 include rail members 251 that are configured to be received within grooves 249a, 249b of pad 225. Rail members 251, in some embodiments, protrude from internal surfaces of side walls 235a, 235b into channel 239 and are sized to fit within grooves 249a, 249b.

Unlike pad 125 and base 129, in some embodiments, pad 225 and base 229 are shaped and sized such that lower surfaces 257a, 257b of rail members 251 and bearing surfaces 259a, 259b of grooves 249a, 249b are able to come into contact. In some embodiments, these surfaces are able to come into contact when, for example, a force F is applied against grasping surface 241 of pad 225. Force F, for example, may be the force applied against grasping surface 241 by blade 13 (not shown) or the target tissue during use. In some embodiments, a distance between top surface 255 of pad 225 and the internal surface of top wall 237 of base 229 may be greater than the distance between lower surfaces 257a, 257b of rail members 251 and bearing surfaces 259a, 259b of grooves 249a, 249b. In some embodiments, pad 225 is moveable from a first position in which bearing surfaces 259a, 259b are not in contact with rail members 251 to a second position in which bearing surfaces 259a, 259b are in contact with rail members 251, for example, in response to force F being applied against grasping surface 241 of pad 225. In some embodiments, lower surfaces 257a, 257b of rail members 251 and bearing surfaces 259a, 259b of grooves 249a, 249b are always in contact when pad 225 is received within base 229. In some embodiments, contact between lower surfaces 257a, 257b of rail members 251 and bearing surfaces 259a, 259b of grooves 249a, 249b allows for improved heat transfer (depicted by the dashed arrow lines) from pad 225 to rail members 251. In some embodiments, pad 225 may be sized such that a small gap is present between the lateral sides of pad 225 and the internal surfaces of side walls 235a, 235b. The small gap, in some embodiments, may help facilitate insertion of pad 225 into base 229. In some embodiments, for example, the internal surfaces of side walls 235a, 235b may be spaced from pad 225 by 0 mm (no gap) up to 0.15 mm.

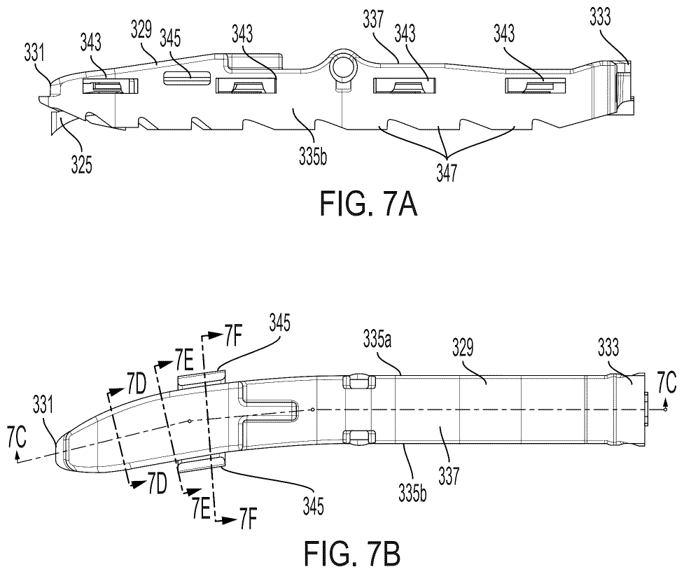

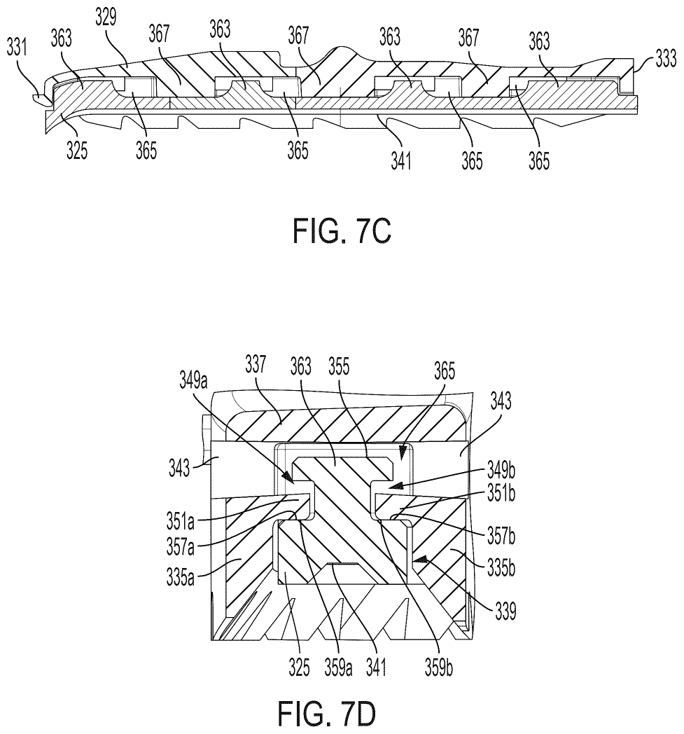

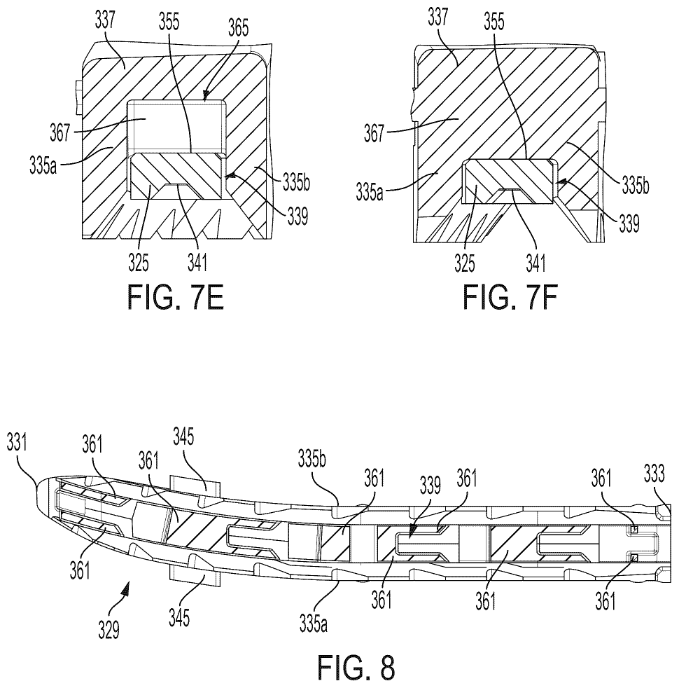

FIGS. 7A-7F illustrate views of a base 329 and pad 325 according to further embodiments of the present disclosure. Base 329 and pad 325 may be used for base 29 and pad 25 in grasping instrument 10 of ultrasonic treatment device 1. More particularly, FIG. 7A shows a side view of base 329 and pad 325 received therein. FIG. 7B shows a top view of base 329 and pad 325. FIG. 7C shows a cross-sectional view of base 329 and pad 325 along the plane designated by line 7C-7C in FIG. 7B. FIG. 7D shows a cross-sectional view of base 329 and pad 325 along the plane designated by line 7D-7D in FIG. 7B. FIG. 7E shows a cross-sectional view of base 329 and pad 325 along the plane designated by line 7E-7E in FIG. 7B. FIG. 7F shows a cross-sectional view of base 329 and pad 325 along the plane designated by line 7F-7F in FIG. 7B.

As shown in the illustrated embodiments of FIGS. 7A-7F, base 329 includes a distal end 331 and a proximal end 333 that is opposite of distal end 331. Base 329, in some embodiments, further includes a pair of opposite side walls 335a, 335b, and a top wall 337 that may extend from distal end 331 to proximal end 333. Base 329 may be constructed from a metal or metal alloy. In some embodiments, side walls 335a, 335b may have substantially the same thicknesses. In some embodiments, base 329 includes an inner channel 339 (see, e.g., FIG. 7D) that is defined by at least one wall such as side walls 335a, 335b, and top wall 337. In some embodiments, channel 339 is sized and configured for receiving pad 325 therein. In some embodiments, channel 339 is open opposite of top wall 337 such that a grasping surface 341 of pad 325 may be exposed while pad 325 is received in channel 339. At least a portion of grasping surface 341, in some embodiments, may be configured to contact the target tissue during use. In some embodiments, grasping surface 341 may also come into contact with blade 13 when grasping instrument 10 is in the closed configuration. Pad 325 further includes a top surface 355 that is opposite grasping surface 341. In some embodiments, portions of top surface 355 may abut against an internal surface of top wall 337. FIG. 8 is a bottom view of base 329 showing portions 361 that may contact the pad according to some embodiments.

Referring again to FIGS. 7A-7F, in some embodiments, base 329 includes one or more openings 343. In some embodiments, each of side walls 335a, 335b include one or more openings 343. As shown in FIG. 7A, openings 343 are elongated openings arranged linearly along a longitudinal axis of base 329. In some embodiments, the one or more openings extend through the entire thickness of side wall 335a or 335b and communicate with channel 339, as best shown in FIG. 7D. In some embodiments, base 329 includes one or more external projections 345. In some such embodiments, the one or more external projections 345 are received within openings or recesses on distal cover member 27a of jaw 21 (see FIG. 3) to help secure base 329 to distal cover member 27a. In some embodiments, each of side walls 335a, 335b include at least one external projection 345 that extends from an exterior portion of side wall 335a, 335b. In some embodiments, a bottom edge of each of side walls 335a, 335b may optionally include one or more teeth 347. Teeth 347, in some embodiments, may assist jaw 21 in grasping and holding the target tissue against blade 13 during use.

Pad 325, in some embodiments, is sized to extend from distal end 331 to proximal end 333 of base 329 within channel 339. Pad 325 may be constructed from a polymer material, for example, polytetrafluoroethylene (PTFE) or other polymer resin, according to some embodiments. In some embodiments, unlike pad 125, pad 325 may not have a generally constant cross-sectional shape along its length, as shown in FIGS. 7C-7F. In some embodiments, the cross-sectional shape of pad 325 may, however, be generally symmetric (e.g., reflection symmetry) along its length. In some embodiments, pad 325 includes portions having different heights between top surface 355 and grasping surface 341. In some embodiments, pad 325 includes a plurality of discontinuous protrusions 363, as best shown in FIGS. 7C and 7D. In some embodiments, protrusions 363 extend from a side of pad 325 that is opposite of grasping surface 341. In some embodiments, each protrusion 363 is configured to be received by a separate slot 365 of base 329. Slots 365 may communicate with openings 343 according to some embodiments. Slots 365 may also communicate with channel 339. As shown in FIGS. 7C, 7E, and 7F, in some embodiments, separating adjacent slots 365 are thickened portions 367 of top wall 337. Thickened portions 367, in some embodiments, have a greater thickness than adjacent portions of top wall 337 (e.g., by at least 30% to 50%). In some embodiments, the thickened portions 367 of top wall 337 are configured as heat sinks for absorbing heat from pad 325. In some embodiments, thickened portions 367 extend into channel 339 and are configured to contact portions of pad 325 that are between protrusions 363. For example, in some embodiments, thickened portions 367 are configured to contact a surface of pad 325 that is opposite of grasping surface 341 (e.g., top surface 355). In some embodiments, the thickened portions 367 of top wall 337 may serve to increase the heat capacity of base 329.

Pad 325, in some embodiments, includes grooves 349a, 349b (see, e.g., FIG. 7D) located on opposing lateral sides of pad 325. Unlike pad 125, in some such embodiments, grooves 349a, 349b do not extend along the entire length or a majority of the length of pad 325. Rather, in some embodiments, grooves 349a, 349b are located only on intermittent protrusions 363 of pad 325. In some embodiments, side walls 335a, 335b of base 329 include rail members 351a, 351b that are configured to be received within grooves 349a, 349b of pad 325. Rail members 351a, 351b, in some embodiments, protrude from internal surfaces of side walls 335a, 335b and are sized to fit within grooves 349a, 349b. In some embodiments, rail members 351a, 351b are only present within slots 365 of base 329.

As further shown in FIG. 7D, in some embodiments pad 325 and base 329 are shaped and sized such that lower surfaces 357a, 357b of rail members 351a, 351b and bearing surfaces 359a, 359b of grooves 349a, 349b are able to come into contact. In some embodiments, these surfaces are able to come into contact when, for example, a force is applied against grasping surface 341 of pad 325. The force may be the force applied against grasping surface 341 by blade 13 (not shown) or the target tissue during use, for instance. In some embodiments, within slot 365 of base 329, a distance between top surface 355 of pad 325 and the internal surface of top wall 337 of base 329 may be greater than the distance between lower surfaces 357a, 357b of rail members 351a, 351b and bearing surfaces 359a, 359b of grooves 349a, 349b. In some embodiments, pad 325 is moveable from a first position in which bearing surfaces 359a, 359b are not in contact with rail members 351a, 351b to a second position in which bearing surfaces 359a, 359b are in contact with rail members 351a, 351b, for example, in response to a force being applied against grasping surface 341 of pad 325. In some embodiments, lower surfaces 357a, 357b of rail members 351a, 351b and bearing surfaces 359a, 359b of grooves 349a, 349b may always be in contact when pad 325 is received within base 329. In some embodiments, contact between lower surfaces 357a, 357b of rail members 351a, 351b and bearing surfaces 359a, 359b of grooves 349a, 349b allows for improved heat transfer from pad 325 to rail members 351a, 351b and base 329. In some embodiments, pad 325 may be sized such that a small gap is present between the lateral sides of pad 325 and the internal surfaces of side walls 335a, 335b. The small gap, in some embodiments, may help facilitate insertion of pad 325 into base 329. In some embodiments, for example, the internal surfaces of side walls 335a, 335b may be spaced from pad 325 by 0 mm (no gap) up to 0.15 mm.

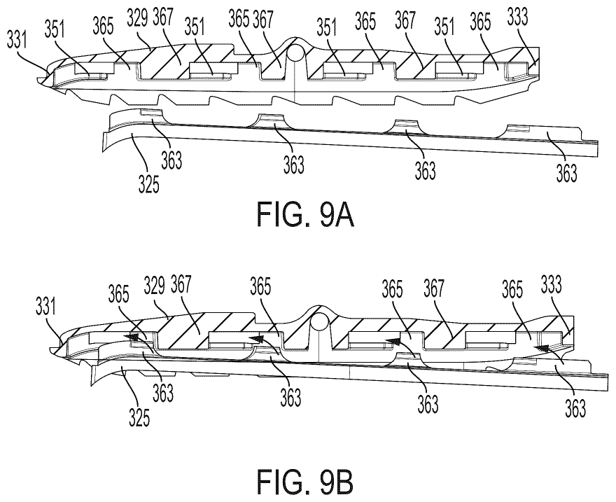

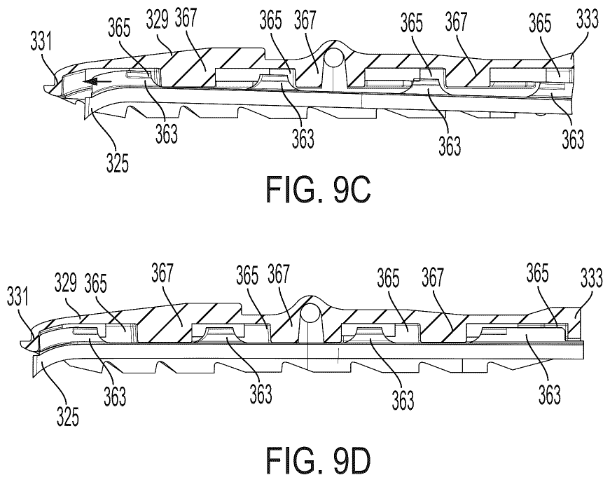

FIGS. 9A-9D illustrate cross-sectional views showing sequential steps for assembling pad 325 with base 329 according to some embodiments. In some embodiments, pad 325 is positioned relative to base 329 such that protrusions 363 of pad 325 are aligned with slots 365 of base 329. In some embodiments, each protrusion 363 is aligned with a separate slot 365 of base 329. In some embodiments, pad 325 is moved relative to base 329 such that protrusions 363 are introduced into slots 365. In some embodiments, pad 325 is moved relative to base 329 in a first direction that may be oblique or perpendicular to the longitudinal axis of base 329 to introduce protrusions into slots 365 (e.g., oblique or perpendicular to a proximal-distal direction). In some embodiments, protrusions 363 are introduced into proximal portions of slots 365. After protrusions 363 are introduced into slots 365, pad 325 is moved in a second direction to engage grooves on protrusions 363 (e.g., grooves 349a, 349b shown in FIG. 7D) with rail members 351 of base. In some embodiments, grooves on protrusions 363 are slid around rail members 351 as pad 325 is moved in the second direction relative to base 329. In some embodiments, the second direction is generally parallel to the longitudinal axis of base 329, e.g., in a proximal-distal direction. In some embodiments, the second direction is generally perpendicular to the first direction. In some embodiments, the second direction is a distal direction (e.g., pad 325 is moved towards distal end 331 of base 329).

Additionally, due to wear and deformation from use, it may be necessary to remanufacture the treatment device by replacing a pad. Accordingly, the pad 325 and base 329 of a treatment device can be remanufactured by removing a used pad from the base by, for example, performing in reverse sequence the steps for assembling the pad and the base (as disclosed herein), and then assembling an unused pad 325 to the base 329. When remanufacturing a treatment device, the used treatment device should first be cleaned, disinfected, and sterilized to ensure a clean condition. Then, the used treatment device can be disassembled to replace one or more parts, such as the pad 325. When replacing the pad 325, a used pad is removed from the used treatment device in reverse order of the steps for assembling the pad 325 and the base 329 as shown and described with respect to FIGS. 9A-9D. Then, an unused pad is assembled in the steps for assembling the pad and the base as shown and described with respect to FIGS. 9A-9D. Finally, after the assembly is completed and the assembled device has been inspected, the device is cleaned, disinfected, and sterilized, followed by packaging, shipping and/or use.

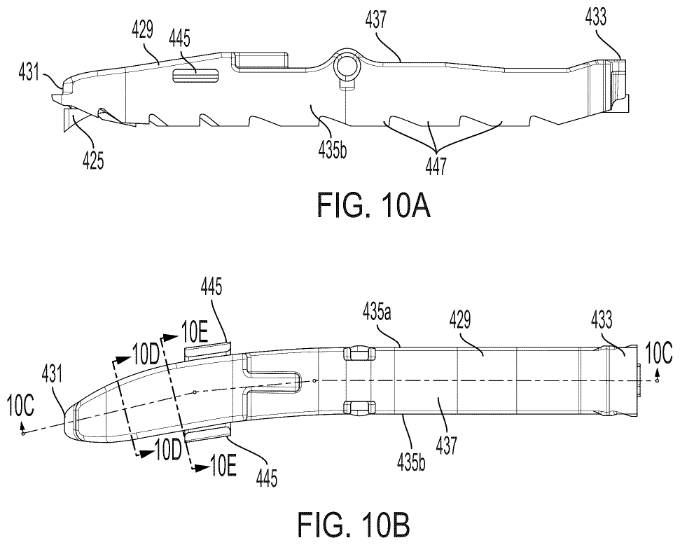

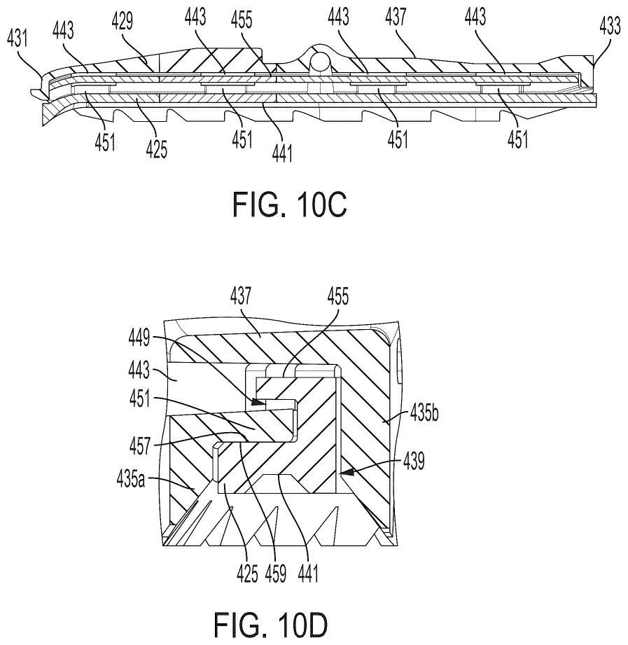

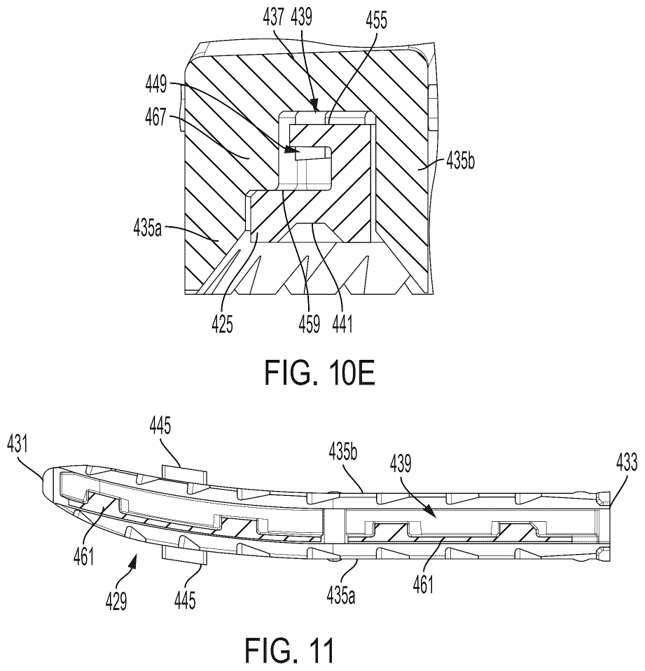

FIGS. 10A-10E illustrate views of a base 429 and pad 425 according to further embodiments of the present disclosure. Base 429 and pad 425 may be used for base 29 and pad 25 in grasping instrument 10 of ultrasonic treatment device 1. More particularly, FIG. 10A shows a side view of base 429 and pad 425 received therein. FIG. 10B shows a top view of base 429 and pad 425. FIG. 10C shows a cross-sectional view of base 429 and pad 425 along the plane designated by line 10C-10C in FIG. 10B. FIG. 10D shows a cross-sectional view of base 429 and pad 425 along the plane designated by line 10D-10D in FIG. 10B. FIG. 10E shows a cross-sectional view of base 429 and pad 425 along the plane designated by line 10E-10E in FIG. 10B.

As shown in the illustrated embodiments of FIGS. 10A-10E, base 429 includes a distal end 431 and a proximal end 433 that is opposite of distal end 431. Base 429, in some embodiments, further includes a pair of opposite side walls 435a, 435b, and a top wall 437 that may extend from distal end 431 to proximal end 433. Base 429 may be constructed from a metal or metal alloy. In some embodiments, side walls 435a, 435b may have different thicknesses. In some embodiments, base 429 includes an inner channel 439 that is defined by at least one wall such as side walls 435a, 435b, and top wall 437. In some embodiments, channel 439 is sized and configured for receiving pad 425 therein. In some embodiments, channel 439 is open opposite of top wall 437 such that a grasping surface 441 of pad 425 may be exposed while pad 425 is received in channel 439. At least a portion of grasping surface 441, in some embodiments, may be configured to contact the target tissue during use. In some embodiments, grasping surface 441 may also come into contact with blade 13 when grasping instrument 10 is in the closed configuration. Pad 425 further includes a top surface 455 that is opposite grasping surface 441. In some embodiments, portions of top surface 455 may abut against an internal surface of top wall 437. FIG. 11 is a bottom view of base 429 showing portions 461 that may contact the pad according to some embodiments.

Referring again to FIGS. 10A-10E, in some embodiments, base 429 includes one or more openings 443. In some embodiments, only one of side walls 435a, 435b includes the one or more openings 443. In the illustrated embodiment, only side wall 435a includes the one or more openings 443. In other embodiments, only side wall 435b includes the one or more openings 443. As shown in FIG. 10C, openings 443 are elongated openings arranged linearly along a longitudinal axis of base 429. In some embodiments, the one or more openings 443 extend through the entire thickness of side wall 435a and communicate with channel 439, as best shown in FIG. 10D. In some embodiments, base 429 further includes one or more external projections 445. In some such embodiments, the one or more external projections 445 are received within openings or recesses on distal cover member 27a of jaw 21 (see FIG. 3) to help secure base 429 to distal cover member 27a. In some embodiments, each of side walls 435a, 435b include at least one external projection 445 that extends from an exterior portion of side wall 435a, 435b. In some embodiments, a bottom edge of each of side walls 435a, 435b may optionally include one or more teeth 447. Teeth 447, in some embodiments, may assist jaw 21 in grasping and holding the target tissue against blade 13 during use.

Pad 425, in some embodiments, is sized to extend from distal end 431 to proximal end 433 of base 429 within channel 439. Pad 425 may be constructed from a polymer material, for example, polytetrafluoroethylene (PTFE) or other polymer resin, according to some embodiments. In some embodiments, except for certain end portions and discounting any surface texturing, pad 425 may have a generally constant cross-sectional shape along its length, or at least a majority of its length. In some embodiments, the cross-sectional shape of pad 425 may be asymmetric, as shown in FIGS. 10D and 10E.

Pad 425 may form a sliding joint with base 429 according to some embodiments. In some embodiments, pad 425 includes only single groove 449 located one side of pad 425. Unlike pad 125, for example, pad 425 in some embodiments includes only a single groove 449. In the illustrated embodiment, groove 449 is located on the side of pad 425 that faces side wall 435a. In some embodiments, groove 449 extends along the entire length or at least a majority of the length of pad 425. In some embodiments, only side wall 435a of base 429 includes a rail member 451 that is configured to be received within groove 449 of pad 425, respectively. Rail member 451, in some embodiments, protrudes from an internal surface of side walls 435a into channel 439 and are sized to fit within groove 449 to form a sliding fit. In some embodiments, rail member 451 extends substantially along the entire length of side wall 435a. In other embodiments, side wall 435a includes a plurality of rail members 451 that are located at different positions along the length of side wall 435a. For example, as shown in FIG. 10C, side wall 435a may include a separate rail member 451 below each opening 443.

As further shown in FIG. 10D, in some embodiments pad 425 and base 429 are shaped and sized such that lower surface 457 of rail member 451 and bearing surface 459 of groove 449 are able to come into contact. In some embodiments, these surfaces are able to come into contact when, for example, a force is applied against grasping surface 441 of pad 425. The force may be the force applied against grasping surface 441 by blade 13 (not shown) or the target tissue during use, for instance. In some embodiments, a distance between top surface 455 of pad 425 and the internal surface of top wall 437 of base 429 may be greater than the distance between lower surface 457 of rail member 451 and bearing surface 459 of groove 449. In some embodiments, pad 425 is moveable from a first position in which bearing surface 459 is not in contact with rail member 451 to a second position in which bearing surface 459 is in contact with rail member 451, for example, in response to a force being applied against grasping surface 441 of pad 425. In some embodiments, lower surface 457 of rail member 451 and bearing surface 459 of groove 449 may always be in contact when pad 425 is received within base 429. In some embodiments, contact between lower surface 457 of rail member 451 and bearing surface 459 of groove 449 allows for improved heat transfer from pad 425 to rail member 451 and base 429.

In further embodiments, portions of side wall 435a may have increased thickness. In some embodiments, certain portions of side wall 435a located between openings 443 may have increased thickness. As shown in FIG. 10E, in some embodiments, side wall 435a includes one or more thickened portions 467. Thickened portions 467, in some embodiments, have a greater thickness than adjacent portions of side wall 435a (e.g., by at least 30% to 50%). In some embodiments, thickened portions 467 of side wall 435a have a thickness greater than a thickness of opposite side wall 435b (e.g., by at least 30% to 50%). In some embodiments, the thickened portion 467 of side wall 435a are configured as heat sinks for absorbing heat from pad 425. In some embodiments, side wall 435a includes a plurality of rail members 451 (see, e.g., FIG. 10C), and thickened portions 467 are located at positions between the rail members 451. In some embodiments, thickened portions 467 extend into channel 439 and are configured to contact portions of pad 425, for example, bearing surface 459 of groove 449. In some embodiments, thickened portions 467 are configured to contact a surface of pad 425 that is opposite of grasping surface 441. In some embodiments, thickened portions 467 of side wall 435a may serve to increase the heat capacity of base 429. In some embodiments, pad 425 may be sized such that a small gap is present between the lateral sides of pad 425 and the internal surfaces of side walls 435a, 435b. The small gap, in some embodiments, may help facilitate insertion of pad 425 into base 429. In some embodiments, for example, the internal surfaces of side walls 435a, 435b may be spaced from pad 425 by 0 mm (no gap) up to 0.15 mm.

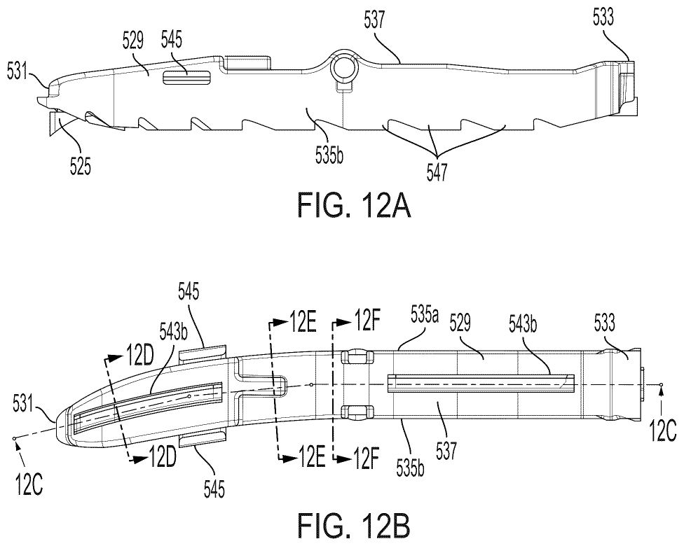

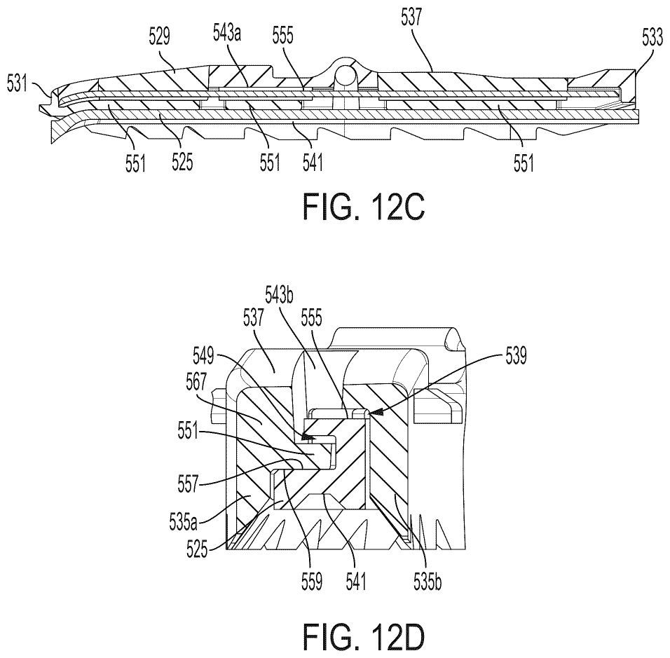

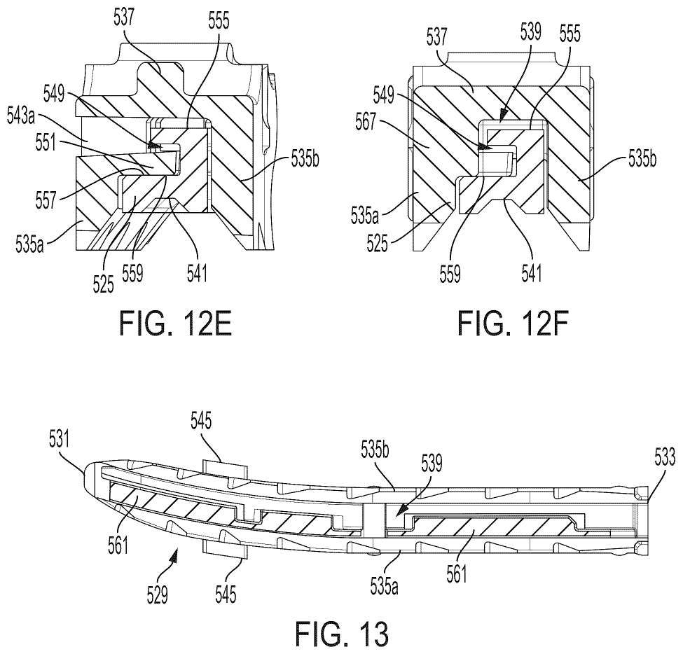

FIGS. 12A-12F illustrate views of a base 529 and pad 525 according to further embodiments of the present disclosure. Base 529 and pad 525 may be used for base 29 and pad 25 in grasping instrument 10 of ultrasonic treatment device 1. More particularly, FIG. 12A shows a side view of base 529 and pad 525 received therein. FIG. 12B shows a top view of base 529 and pad 525. FIG. 12C shows a cross-sectional view of base 529 and pad 525 along the plane designated by line 12C-12C in FIG. 12B. FIG. 12D shows a cross-sectional view of base 529 and pad 525 along the plane designated by line 12D-12D in FIG. 12B. FIG. 12E shows a cross-sectional view of base 529 and pad 525 along the plane designated by line 7E-12E in FIG. 12B. FIG. 12F shows a cross-sectional view of base 529 and pad 525 along the plane designated by line 12F-12F in FIG. 12B.

As shown in the illustrated embodiments of FIGS. 12A-12F, base 529 includes a distal end 531 and a proximal end 533 that is opposite of distal end 531. Base 529, in some embodiments, further includes a pair of opposite side walls 535a, 535b, and a top wall 537 that may extend from distal end 531 to proximal end 533. Base 529 may be constructed from a metal or metal alloy. In some embodiments, side walls 535a, 535b may have different thicknesses. In some embodiments, base 529 includes an inner channel 539 that is defined by at least one wall such as side walls 535a, 535b, and top wall 537. In some embodiments, channel 539 is sized and configured for receiving pad 525 therein. In some embodiments, channel 539 is open opposite of top wall 537 such that a grasping surface 541 of pad 525 may be exposed while pad 525 is received in channel 539. At least a portion of grasping surface 541, in some embodiments, may be configured to contact the target tissue during use. In some embodiments, grasping surface 541 may also come into contact with blade 13 when grasping instrument 10 is in the closed configuration. Pad 525 further includes a top surface 555 that is opposite grasping surface 541. In some embodiments, portions of top surface 555 may abut against an internal surface of top wall 537. FIG. 13 is a bottom view of base 529 showing portions 561 that may contact the pad according to some embodiments.

Referring again to FIGS. 12A-12F, in some embodiments, base 529 includes one or more side openings 543a. In some embodiments, only one of side walls 535a, 535b includes the one or more side openings 543a. In the illustrated embodiment, only side wall 535a includes the one or more side openings 543a. In other embodiments, only side wall 535b includes the one or more openings 543a. In some embodiments, the one or more side openings 543a extend through the entire thickness of side wall 535a and communicate with channel 539, as best shown in FIG. 12E. In some embodiments, base 529 includes one or more top openings 543b (see, e.g., FIGS. 12B and 12D) located in top wall 537. Top opening 543b, in some embodiments, extends through the entire thickness of top wall 537 and communicates with channel 539. Side opening 543a and top opening 543b may each be elongated along a longitudinal axis of base 529.

In some embodiments, base 529 further includes one or more external projections 545. In some such embodiments, the one or more external projections 545 are received within openings or recesses on distal cover member 27a of jaw 21 (see FIG. 3) to help secure base 529 to distal cover member 27a. In some embodiments, each of side walls 535a, 535b include at least one external projection 545 that extends from an exterior portion of side wall 535a, 535b. In some embodiments, a bottom edge of each of side walls 535a, 535b may optionally include one or more teeth 547. Teeth 547, in some embodiments, may assist jaw 21 in grasping and holding the target tissue against blade 13 during use.

Pad 525, in some embodiments, is sized to extend from distal end 531 to proximal end 533 of base 529 within channel 539. Pad 525 may be constructed from a polymer material, for example, polytetrafluoroethylene (PTFE) or other polymer resin, according to some embodiments. In some embodiments, except for certain end portions and discounting any surface texturing, pad 525 may have a generally constant cross-sectional shape along its length, or at least a majority of its length. In some embodiments, the cross-sectional shape of pad 525 may be asymmetric, as shown in FIGS. 12D-12F.

Pad 525 may form a sliding joint with base 529 according to some embodiments. In some embodiments, pad 525 includes a groove 549 located on one side of pad 525. Unlike pad 125, for example, pad 525 in some embodiments includes only a single groove 549. In the illustrated embodiment, groove 549 is located on the side of pad 525 that faces side wall 535a. In some embodiments, groove 549 extends along the entire length or at least a majority of the length of pad 525. In some embodiments, only side wall 535a of base 529 includes a rail member 551 that is configured to be received within groove 549 of pad 525, respectively. Rail member 551, in some embodiments, protrudes from an internal surface of side walls 535a into channel 539 and is sized to fit within groove 549 to form a sliding fit. In some embodiments, rail member 551 extends substantially along the entire length of side wall 535a. In other embodiments, side wall 535a includes a plurality of rail members 551 that are located at different positions along the length of side wall 535a. For example, as shown in FIGS. 12C-12E, side wall 535a may include a separate rail member 551 below each side or top opening 543a, 543b.

As further shown in FIGS. 12D and 12E, in some embodiments pad 525 and base 529 are shaped and sized such that lower surface 557 of rail member 551 and bearing surfaces 559 of groove 549 are able to come into contact. In some embodiments, these surfaces are able to come into contact when, for example, a force is applied against grasping surface 541 of pad 525. The force may be the force applied against grasping surface 541 by blade 13 (not shown) or the target tissue during use, for instance. In some embodiments, a distance between top surface 555 of pad 525 and the internal surface of top wall 537 of base 529 may be greater than the distance between lower surface 557 of rail member 551 and bearing surface 559 of groove 549. In some embodiments, pad 525 is moveable from a first position in which bearing surface 559 is not in contact with rail member 551 to a second position in which bearing surface 559 is in contact with rail member 551, for example, in response to a force being applied against grasping surface 541 of pad 525. In some embodiments, lower surface 557 of rail member 551 and bearing surface 559 of groove 549 may always be in contact when pad 525 is received within base 529. In some embodiments, contact between lower surface 557 of rail member 551 and bearing surface 559 of groove 549 allows for improved heat transfer from pad 525 to rail member 551 and base 529.

In further embodiments, portions of side wall 535a may have increased thickness. In some embodiments, certain portions of side wall 535a bordering top openings 543b may have increased thickness. In some embodiments, a majority of side wall 535a includes increased thickness. As shown in FIGS. 12D and 12F, in some embodiments, side wall 535a includes one or more thickened portions 567. Thickened portions 567, in some embodiments, have a greater thickness than adjacent portions of side wall 535a (e.g., by at least 30% to 50%). In some embodiments, thickened portions 567 of side wall 535a have a thickness greater than a thickness of opposite side wall 535b (e.g., by at least 30% to 50%). In some embodiments, the one or more thickened portions 567 of side wall 535a are configured as heat sinks for absorbing heat from pad 525. In some embodiments, the one or more thickened portions 567 are configured to contact portions of pad 525, for example, bearing surface 559 of groove 549. In some embodiments, rail member 551 extends from a thickened portion 567 of side wall 535a (see, e.g., FIG. 12D). In some embodiments, thickened portions 567 are configured to contact a surface of pad 525 that is opposite of grasping surface 541. In some embodiments, thickened portions 567 are configured to contact bearing surface 559 (see, e.g., FIG. 12F). In some embodiments, the one or more thickened portions 567 of side wall 535a may serve to increase the heat capacity of base 529. In some embodiments, pad 525 may be sized such that a small gap is present between the lateral sides of pad 525 and the internal surfaces of side walls 535a, 535b. The small gap, in some embodiments, may help facilitate insertion of pad 525 into base 529. In some embodiments, for example, the internal surfaces of side walls 535a, 535b may be spaced from pad 525 by 0 mm (no gap) up to 0.15 mm.

While certain embodiments of the present disclosure have been described in connection with specific instruments and treatment procedures, embodiments described herein are not necessarily limited to these specific uses. For example, the various pads and bases described herein are not necessarily limited to use with ultrasonic treatment device 1. Other ultrasonic devices, non-ultrasonic devices, and grasping instruments may also benefit from using components described herein.

It should be understood that various changes, substitutions, and alterations can be made herein without departing from the spirit and scope of the invention as defined by the appended claims. It should also be apparent that individual elements identified herein as belonging to a particular embodiment may be included in other embodiments of the invention. Moreover, the scope of the present application is not intended to be limited to the particular embodiments of the process, machine, manufacture, and composition of matter, means, methods and steps described in the specification. As one of ordinary skill in the art will readily appreciate from the disclosure herein, processes, machines, manufacture, composition of matter, means, methods, or steps, presently existing or later to be developed that perform substantially the same function or achieve substantially the same result as the corresponding embodiments described herein may be utilized according to the present invention.

What is claimed is:

1. An ultrasonic treatment device, comprising:
a blade; and
a jaw configured to open and close relative to the blade,
wherein the jaw includes:
a base including:
a top wall including a first base-thickened portion and a first base-thinned portion, wherein the first base-thickened portion is thicker than the first base-thinned portion,
a plurality of side walls, and
an inner channel defined by the top wall and the plurality of side walls, and
a pad located in the inner channel, the pad including:
a grasping surface facing the blade, and
a top surface opposed to the grasping surface, the top surface including a first pad-thickened portion and a first pad-thinned portion, wherein
the first pad-thickened portion is thicker than the first pad-thinned portion,
wherein the first base-thickened portion abuts the first pad-thinned portion, and
wherein the first pad-thickened portion is located under the first base-thinned portion.

2. The ultrasonic treatment device of claim 1, wherein the first base-thickened portion is adjacent to the first base-thinned portion.

3. The ultrasonic treatment device of claim 1, wherein the first base-thickened portion and the first base-thinned portion are aligned along a longitudinal axis of the base.

4. The ultrasonic treatment device of claim 1, wherein the first pad-thickened portion is adjacent to the first pad-thinned portion.

5. The ultrasonic treatment device of claim 1, wherein the first pad-thickened portion and the first pad-thinned portion are aligned along a longitudinal axis of the pad.

6. The ultrasonic treatment device of claim 1, wherein, in a direction of a longitudinal axis of the jaw, the first pad-thickened portion is spaced apart from the first base-thinned portion.

7. The ultrasonic treatment device of claim 1, wherein the top wall includes a second base-thickened portion and a second base-thinned portion, and wherein the top surface includes a second pad-thickened portion and a second pad-thinned portion.

8. The ultrasonic treatment device of claim 7, wherein, in a direction of a longitudinal axis of the jaw, the first based-thinned portion is located between the first base-thickened portion and the second base-thickened portion.

9. The ultrasonic treatment device of claim 7, wherein, in a direction of a longitudinal axis of the jaw, the second base-thickened portion is located between the first base-thinned portion and the second base-thinned portion.

10. The ultrasonic treatment device of claim 7, wherein, in a direction of a longitudinal axis of the jaw, the second pad-thinned portion is located between the first pad-thickened portion and the second pad-thickened portion.

11. The ultrasonic treatment device of claim 7, wherein, in a direction of a longitudinal axis of the jaw, the first pad-thickened portion is located between the first pad-thinned portion and the second pad-thinned portion.

12. The ultrasonic treatment device of claim 7, wherein, in a direction of a longitudinal axis of the jaw, the first base-thickened portion is spaced part from the first pad-thickened portion.

13. The ultrasonic treatment device of claim 12, wherein, in the direction of the longitudinal axis of the jaw, the second base-thickened portion is spaced part from the first pad-thickened portion.

14. The ultrasonic treatment device of claim 1, wherein the first base-thickened portion is configured as a heat sink.
